(19) United States
(12) Patent Application Publication
(10) Pub. No.: US 2026/0151566 A1
(43) Pub. Date: Jun. 4, 2026

(54) SYRINGE BASED EMERGENCY MEDICAL PREPARATION CONVENIENCE KIT

(71) Applicant: Thorne Intellectual Property Holdings, LLC, Bountiful, UT (US)

(72) Inventors: Kendall Patterick Thorne, Layton, UT (US); Gale Harrison Thorne, Bountiful, UT (US); Gale Harrison Thorne, JR., Bontiful, UT (US)

(73) Assignee: Thorne Intellectual Property Holdings, LLC, Bountiful, UT (US)

(21) Appl. No.: 18/831,353

(22) Filed: Dec. 2, 2024

(51) Int. Cl. A61M 5/178; A61L 2/00; A61M 5/00; A61M 5/31

(52) U.S. Cl. CPC A61M 5/1782; A61M 5/002; A61M 5/31; A61L 2103/05; A61M 2005/3118; A61M 2205/75

(57) ABSTRACT

A convenience kit is disclosed which can be used for filling pre-capped syringes with medicine sterilized by a single pathway filter assembly into a closed and sealed plastic bag which provides a protective housing in which the syringes are uncapped, filled and recapped in sterile environs and prepared for use in a potentially contaminating environment. A capet (a cap engaging tool) for facile in-bag manipulation associated with uncapping and capping each syringe is also disclosed.

### FIELD OF INVENTION

Inventions recorded herein were inspired by the tragic results of Hurricane Helene as effected upon North Carolina in October 2024.

As reported in BECKER'S HOSPITAL REVIEW (circa 2024):

An immense Baxter International Plant located in North Carolina which produced large amounts of IV solutions for nationwide use was rendered out of service. Even though IV solution shortages have long existed before Hurricane Helene, this event has severely compounded difficulties in providing adequate quantities of IV solutions, yielding potential patient risk to those who are in need of such medicine. The loss of production from the Baxter plant has triggered severe shortages of such solutions as dextrose, lactated Ringers and those used in perenteral dialysis. To attempt to alleviate the shortage, President Biden invoked the Defense Production Act and initiated foreign importing. Even so, attempts to meet current requirements by attempting to acquire additional product from another Baxter plant, B Braun Medical, ICU Medical or Fresenius Kobi, which are already operating at full capacity and receipt of foreign product is considered neither an adequate nor desired solution. (Also, acquisition of product from foreign sources is currently very expensive in both delays and cost.) As well, experts have stated that long-term shortages shall continue unless there is a guarantee of long term profits for companies from production of IV solutions.
As copied from a recent (2024) Federal Drug Administration Brochure:

Sterile injectables continue to be the most common product in shortage. 64% of shortages in 2023 involved sterile injectables. 2023 saw demand increases outpace manufacturing issues and active pharmaceutical ingredient (API) availability as the underlying reason for the shortage.

### Shortages are a Global Issue

Drug manufacturing in more than one facility, and more than one geographic region, can provide agility that reduces the risk of drug shortages and helps with resolution of shortages when they occur. It is important to have alternative to manufacture the drug, or its API, to improve overall supply chain resiliency. (Underlining for emphasis)

It is for reasons so emphasized by the FDA that inventive convenience kit apparatus and methods disclosed herein are anticipated to fill an important role in providing severely needed pharmaceuticals. By providing convenience kits capable of being used locally to produce containers filled with sterilized medicine in quantities which can meet, often enormous needs of a hospital, a very important role can be played in reducing drug shortages. Use of convenience kits made according to inventions disclosed herein can provide an alternative solution to problems expressed supra by producing IV solutions and other drugs, at a level of safety, convenience and responsiveness never before realized.

Recognizing that day-to-day operations in hospitals use immense numbers of bagged and syringe-based products, a convenience kit used as a substitute source must have a capacity to fill and cap a large number of containers. This translates into need for efficacious operation which must be achieved by each kit function. For a kit to be effective, the containers must not only be filled but also capped before being distributed into a potentially contaminating environment.

Generally, it is well known and understood that pre-filled and capped containers (dose bags) with predetermined volumes of properly sterilized medicine are currently most often provided in large quantities by the large, centralized compounding pharmacies which have facilities and trained personnel for providing medicine packaged at a desired volume and Sterility Assurance Level (SAL) for safe and effective medicine delivery. Such medicine may range from being non-hazardous to hazardous, but in all cases must be provided in sealed containers with a predetermined SAL, sufficient dosage and well before a beyond use date (BUD). In other words, medicine must be timely delivered to a point of use, sterilized to a predetermined SAL and securely capped.

Rather than individual dose bags, syringes can be provided instead using a convenience kit made according to the instant inventions disclosed herein, whereby desired sterilizing, filling and capping of syringes with accurate doses of medicine can be provided to achieve a prescribed SAL within the safety of closed and sealed plastic bags which are pre-sterilized before use. Thereby, all specifications of USP Chapter 797, which strictly specifies conditions for preparing of all IV products, can be met and assured, providing product which is usable for providing IV solutions in even remote patient care facilities. By doing so, problems associated with large and centrally disposed facilities being the only current source of so limited IV solutions and other medicine can be effectively solved and anticipated wide distribution of such convenience kits can eliminate the problems created by localized natural or man-made catastrophes which have been found to occur to current large volume producers. By using such convenience kits to provide a medically viable product, the estimated result will also likely be a reduction in overall cost of medicine as medicine used in filling is purchased or formulated directly by the medicine preparing institution.

Chart A, which follows, provides a summary of examples of syringe sizes and fills and associated medicines which can be produced by distributed preparations in convenience kits, distantly deployed from the large centralized facilities as mentioned supra.

### Chart A

The following chart provides examples of medicines which can be sterilized or maintained at a predetermined sterility level when displaced into syringes at desired volumes using convenience kits made according to instant inventions disclosed herein:

| | | | | | Examples of medi- |
|---|---|---|---|---|---|
| Areas of | | | Nominal | | cines or appli- |
| application | Syringe size(s) | | Fill Volume | | cations involved |
| Irrigation | 3 | mL | 0.5 | mL | Moxifloxacin |
| (Eye surgery) | | | | | |
| IV Antibiotics | 10 | mL | nonspecific | | Ancef, |
| | | | | | Cephalospirins, |
| | | | | | Penicillins |
| Anesthetics | 5, 10, 20 | mL | 5 mL, | | Lidocaine, |
| | | | 10 mL, 20 mL | | Ketamine, |
| | | | | | Succinyeloline |
| Anesthesia | 60 | mL | 60 | mL | Non specific |
| Cocktails | | | | | |
| Anesthesia | 1 | mL | 1 | mL | Epidural & spinal |
| (other) | | | | | injections; (in |
| | | | | | Glass syringes) |
| Surgery | 1 | mL | .1 | mL | Mitomycin, |
| | | | | | Pterigrium, |
| | | | | | tissue growth |
| | | | | | inhibitor |
| Pain | 1 to 60 | mL | Up to 60 mL | | Opioides e.g. |
| management | | | | | Morphine, Meperidine |
| | | | | | via PCA Pump |
| IM Pain | 1 | mL | <1 | mL | Opioides e.g. |
| management | | | | | Morphine, |
| | | | | | Meperidine (“Y” |
| | | | | | site injection) |
| Macular | 1 | mL | .07 | mL | Vitreous Substitutes |
| degeneration | | | | | (e.g. Avastin) |
| treatment | | | | | |

### DESCRIPTION OF RELATED ART

As stated supra, medicine provided, as listed in Chart A, is commonly provided as a sterile product in closed and sealed bags filled and provided by large and centralized facilities. Currently, need is greater than capacity of the producing facilities leading to ever-present shortages.

Also, within the IV solution dispensing art, apparatus and methods for using syringes for patient treatment, rather than bags, are well known and are available generally. In other words, methods disclosed for using syringes to dispense IV solutions are well known and presently, readily used in medical institutions. So, the problem addressed herein is not the use of syringes for dispensing IV solutions, but for providing a convenience kit for filling syringes and capping syringes while maintaining sterility.

Regarding convenience kits which provide for sterilizing, filling and capping syringes for use in medical treatment in potentially contaminating environments, examples of such are provided in U.S. Pat. Nos. 10,555,872 B1 and 12,064,394 which instruct capping syringes before removal from a protective plastic bag in which filling is performed. However, there is no known tool, information nor instruction for providing a large number of bag-contained syringes accessible from outside a bag which provides for available caps and capping before filling.

### Table of Definitions

Following is a table of a list of words and phrases with definitions clarifying their use in this document:
at least one, n: one or more.
bag entry technology. n; apparatus and method for controlling fluid influx into a plastic bag generally comprising a sealed and closed plastic bag with a single hole provided for fluid flow in and out of the bag through provided fittings, the hole being surrounded and otherwise closed to fluid flow by a gasket which creates a single pathway permissive to fluid flow only through the fittings and impedes all other fluid communication into and out of the bag, such communication may be with or without flow through a sterilizing filter.
cap, n: a device used to close an otherwise exposed orifice of a syringe or other container to maintain purity and sterility of syringe contents thereby.
cap, v: to affix a capping device to seal and protect contents of a container.
capet, n: an acronym for “cap engaging tool” preferably affixed to a filter assembly for decapping and capping a cap affixed to a syringe.
decap, v: to remove a cap from a syringe
dose syringe, n: a syringe which is filled with medicine prepared for use in a medical procedure.
filling, v: displacement of liquid into a vessel (e.g. a syringe) generally limited to an amount consistent with a desired quantity of medicine to be displaced into the vessel.
filter, n: a sterilizing filter which is considered to sterilize all fluid which is passed there through, such filters generally have a 0.2 micron filtering capability and so filter all fluids until becoming wet when such a filter will not pass gas. This inherent gas blockage feature is commonly used to test filter efficacy by a “bubble” test performed at the end of a liquid sterilizing medical form.
filter assembly, n: a fluid transmission assembly, consisting of a filter and other parts, having a purpose of communicating fluid into a kit bag via a pathway such that only raw medicine derived from a source is dispatched as a sterile product to an internal source vessel disposed in a sterile environment inside a kit bag.
fishing, v: using multiple attempts to displace an item for insertion which requires precise positioning under conditions which do not permit clear visibility.
filling, v: communicating a predetermined volume of medicine into a syringe.
fitting, n: a fluid pathway connecting device, generally a luer connector which is well known in medical art.
hands free, adj: a method of performing a task without direct hand or digital contact.
kit bag(bag), n: a plastic bag, being a part of a convenience kit, used for providing a protected space wherein a closed (sterile) environment disposed therein provides for safe and effective preparation of a prescribed medicine, the bag being made from pliant, preferably transparent material which is strong enough to resist rupturing when used as part of the convenience kit and yet sufficiently pliant to provide for manipulation of associated convenience kit devices affixed to and disposed within the bag to be used in a medicine preparation process, the bag, generally, comprising a single hole through which a filter assembly comprising a sterilizing filter communicates sterilized medicine into the bag, the hole being closed and sealed about a portion of the filter assembly by a gasket disposed between a fitting affixed to a part of the filter assembly inside the bag and an exposed surface of the filter exterior to the hole and the bag to assure maintenance of a predetermined SAL within the bag provided by sterilization before use.
raw medicine, n: medicine, supplied exterior to the bag, which must be sterilized before use.
SAL, n: Sterility Assurance Level.
syringe, n: a device used for transferring and displacing fluid in a medical application; each syringe having an elongated and substantially constant diameter hollow barrel having an opening at one end where through a plunger and associated plunger rod are impelled to displace fluid resulting from forceful contact between an end button on the plunger rod and finger flanges disposed about the end opening, the barrel having a spout at the other end, preferably comprising a male luer fitting and an orifice through which the fluid is displaced in communication with the barrel. For constrained and special applications particular specifications may be placed upon the syringe, such as zero dead space or being free of silicone oil.
tray (syringe tray), n: a planar appliance which provides an array of compartments wherein syringes for use within the kit bag are disposed for access by a capet.

### BRIEF SUMMARY AND OBJECTS OF THE INVENTION

In brief summary, this novel invention alleviates all of the known problems related to providing an emergency medicine preparation convenience kit for syringe filling by providing steps for accessing a single empty, capped syringe, which can be disposed with a plurality of other syringes within a sterile and safe harbor of a plastic kit bag, removing a cap provided affixed to the syringe, affixing the decapped syringe to a filter assembly and communicating a desired quantity of sterilized medicine into the syringe and recapping the syringe while being continuously disposed within a closed and sealed, pre-sterilized plastic bag. For the purposes of decapping and capping each syringe, it is a unique object to provide a cap engagement tool (referenced as “capet” hereafter) for removing a cap provided affixed to the syringe and recapping the syringe after filling.

A kit bag, used for providing a protected space wherein a closed (sterile) environment disposed therein provides for safe and effective preparation of a prescribed medicine,

Accordingly, it is a primary object to provide convenience kits, made according to the present invention, each kit being a self-contained product, comprising a housing formed by a plastic bag provided for retaining interiorly disposed devices in an uncontaminated state, by being pre-disposed and pre-sterilized within the plastic bag which is closed, sealed and sterilized after the devices and syringes are deposited therein.

It is an essential object to sterilize all matter, including raw medicine, which is communicated into the plastic bag to assure maintenance of a desired SAL. Therefore, it is a basic object to provide a medical sterilizing grade filter assembly which affords the only liquid pathway into the bag,

It is a crucial object to provide tools and apparatus for ready access and handling of empty syringes disposed within the bag to alleviate otherwise time and energy wasting activity associated with syringe handling.

It is a very important object to provide for accurately and precisely filling each syringe with prescribed volumes of medicine.

It is a fundamental object to also provide ready access to caps and capping each syringe after syringe filling and before the plastic bag is breached to assure sterility maintenance of so filled syringes when delivered into a potentially contaminating environment. Therefore, it is an important object to provide empty syringes capped and provide apparatus and methods for decapping and recapping as a part of syringe filling.

It is a major object to provide a syringe holding tray with a plurality of compartments being formed and oriented such that each syringe, disposed therein, can be facilely accessed for individual filling as an option for providing syringes organized.

It is a primary object to provide a cap engagement tool (a “capet”) for use in accessing a selected syringe for filling, removing an associated cap prior to syringe filling and for recapping after filling.

It is an important object to provide a method for affixing the capet to the filter assembly to reduce in-bag tool acquisition time and effort.

It is a key object to provide means for communicating with a sufficiently large source of raw medicine such that syringe filling can be performed as a continuous series of steps.

It is also a critical object to perform a “bubble test” after syringe filling is complete to assure each syringe is filled with appropriately sterilized medicine.

These and other objects and features of the present invention will be apparent from the detailed description taken with reference to accompanying drawings.

### BRIEF DESCRIPTION OF THE DRAWINGS

FIG. 1 is a top elevation of a convenience kit made according to inventions disclosed in this U.S. patent application.

FIG. 2 is a cross section of a PRIOR ART bag entry technology used in making convenience kits for sterilizing medicine.

FIG. 3 is a cross section of a bag entry technology which employs a novel cap engaging technology, disclosed herein, to provide a method for decapping and recapping syringes hands free.

FIG. 4 is a perspective of a syringe being picked from a tray using a capet.

FIG. 5 is a perspective of a syringe cap.

FIG. 6 is a perspective of a PRIOR ART capped medical syringe.

FIG. 7 is a perspective of the medical syringe seen in FIG. 6 with cap removed.

FIG. 8 is a side elevation of a capped syringe disposed for displacement into contact with a capet.

FIG. 9 is a perspective of an in-bag facing portion of a capet.

FIG. 10 is a perspective of the opposite side of the capet seen in FIG. 9.

FIG. 11 is a flow chart providing a series of steps required to sterilize, decap, fill, and cap a plurality of syringes, according to the instant invention, originally disposed in a holding tray.

FIG. 12 is a flow chart providing a series of steps involved with sterilizing, decapping, filling and recapping a plurality of syringes according to the instant invention, disposed in a bag without a tray.

FIG. 13 is a perspective showing a method for digitally selecting and handling a syringe for decapping, filling and recapping within a plastic bag.

FIG. 14 is a perspective showing displacement of a cap into a capet:

FIG. 15 is a perspective showing syringe and cap displacement, following cap displacement as seen in FIG. 13, into a capet entry site.

FIG. 16 is a perspective showing cap displacement from an entry site to a cap retention site.

FIG. 17 is a perspective showing syringe displacement from an associated cap disposed within a capet.

FIG. 18 is a perspective showing a syringe (after filling) being displaced for being recapped.

FIG. 19 is a perspective showing a capped, filled syringe being displaced from the capet.

FIG. 20 is a perspective of a medicine source filling assembly comprising raw medicine in a source bag, a peristaltic pump and a long tubular connection to elements of a convenience kit made according to instant inventions disclosed herein.

### DETAILED DESCRIPTION OF THE ILLUSTRATED EMBODIMENTS

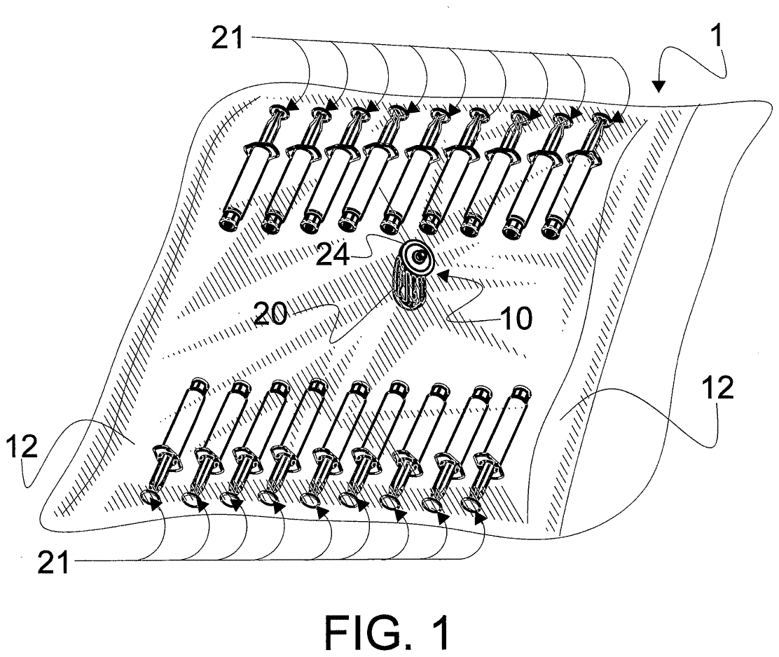

In this description, the term proximal is used to indicate the segment of the device normally closest to the object of the sentence describing its position. The term distal refers to the other end. Reference is now made to the embodiments illustrated in FIGS. 1-20 wherein like numerals are used to designate like parts throughout. Primes of numbers are indicative of like numbered parts which are similar in application and function, but with noted physical differences from the like numbered parts.

Reference is now made to FIG. 1 wherein a convenience kit 1 made according to the instant inventions, disclosed herein, is seen to comprise a PRIOR ART filter assembly 10 affixed to a plastic bag 12 to thereby provide the only fluid pathway into bag 12 when closed and sealed. A cap engagement tool, identified as capet 20 and disposed within bag 12, is seen affixed to filter assembly 10. Also a plurality of capped syringes, each numbered 21 are disposed as part of kit 1 in bag 12.

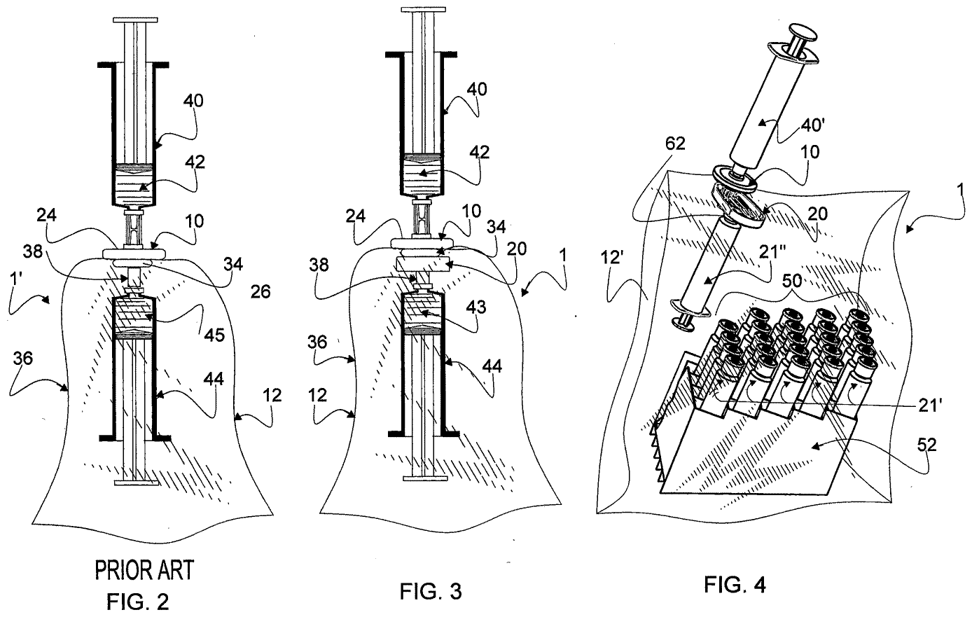

As seen in FIG. 2, PRIOR ART filter assembly 10 comprises a medical grade sterilizing filter 24 which has a male luer fitting (not shown) which is disposed through a hole (also not shown) in a section 36 of bag 12, the hole being closed by a gasket 34 held fast to close the hole by a female/female fitting 38 disposed against gasket 34. Details of a filter assembly similar in form and function to filter assembly 10 are disclosed in detail in U.S. Pat. No. 10,555,872. Generally, an exterior medicine source container, in this case, syringe 40 is affixed to filter assembly 10 to communicate raw medicine 42 through filter assembly 10 which is thereby sterilized to provide dispensing of sterile medicine 45 into an in-bag container, (in this case, a syringe 44).

Before proceeding with detailed apparatus and method disclosure, it is considered expedient to provide a disclosure of terms and identifiers used in this specification for syringes and, other kit parts. A conventional, capped syringe 21 is seen in FIG. 6. Syringe 21 is seen to comprise a barrel 50, a plunger rod 52 affixed to a plunger disposed within barrel 50 (but not shown) and a cap 56 affixed at a spout end of barrel 50. At the end of barrel 50 where plunger rod 52 is exposed is a pair of finger flanges, both numbered 54.

Syringe 21 is seen in FIG. 7 with cap 56 removed exposing a syringe spout 58. Spout 58 preferably has the form and function of a luer fitting.

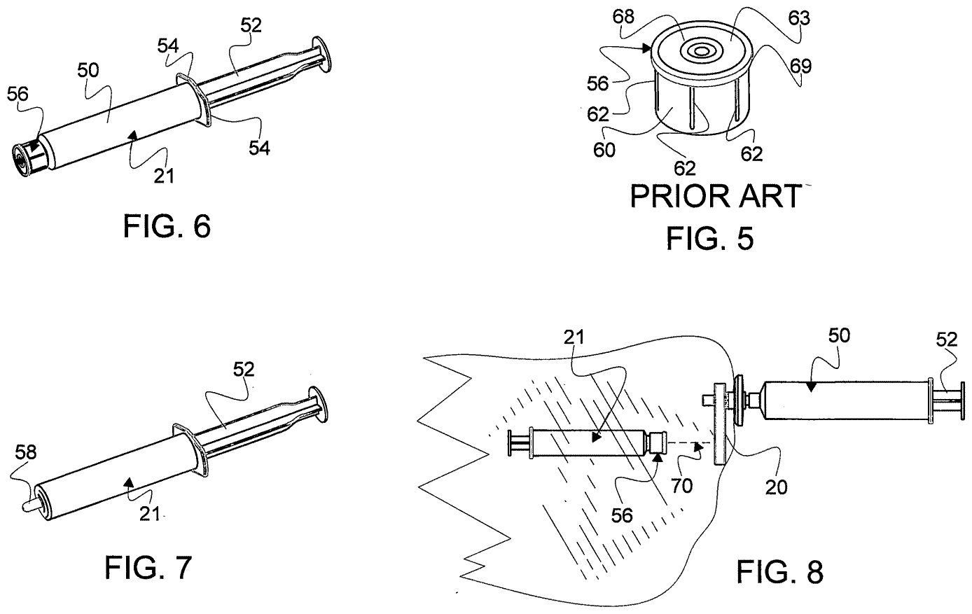

A cap 56, seen in FIG. 5, has a relatively smooth cylindrical body 60 interrupted by vertical ribs, each numbered 62. A top (syringe capping) portion 63 of cap 56 comprises a circular pattern 68 which extends outward from body 60 to provide a circular ridge 69 for facile digital cap acquisition.

In FIG. 8, a capped. empty syringe 21 is disposed for engagement with a capet 20 via dashed line 70 for decapping. Details of modes of syringe and capet 20 operation are made evident in FIGS. 15-20.

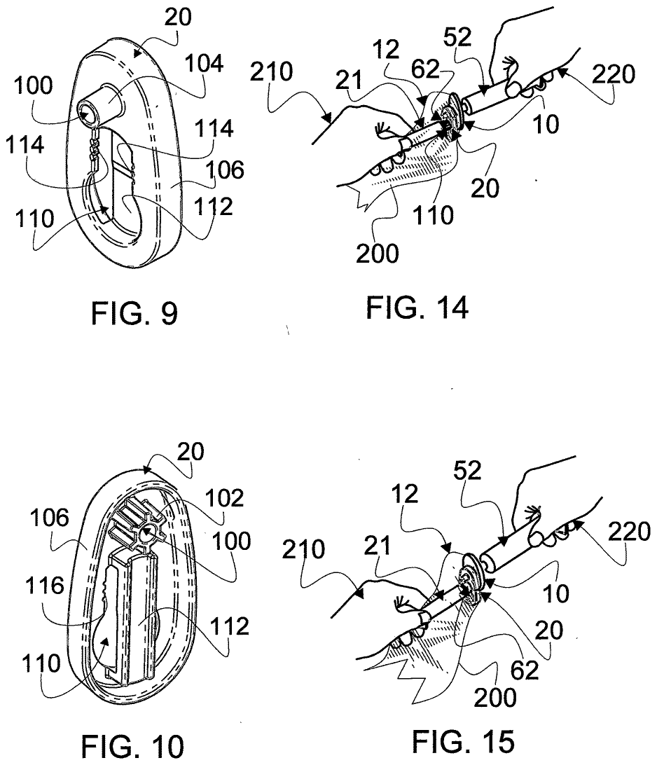

A capet 20 is seen in two views in FIGS. 9 and 10. Capet 20 is seen in an in-bag syringe and cap 56 facing side in FIG. 9 and in a filter assembly 10 facing side in FIG. 10. An orifice 100, seen both views provides for introducing and affixing capet 20 to filter assembly 10 which is used for communicating sterilized medicine from filter 24 to a syringe, such as syringe 21. Connector part 102 is preferably a luer fitting and, as well, connector part 104 is also a luer fitting for interfacing with filter assembly 10. A rim 106 provides a rail for displacing and guiding cap 56 into confinement in capet 20.

As seen FIG. 9 a cap entry orifice 110 is sized and shaped for introduction of a cap 56 and restricted by a back plate 112. Back plate 112 limits depth of such introduction and then is permissive to cap displacement upward until teeth 114 are in contact with cap 56 to thereby engage ribs 62 of cap 56. Such engagement, resists cap rotation and by contact with ribs 68, permitting an attached syringe to be rotated and displaced from cap 56. Note that cap 56 is then held in place until physically retrieved.

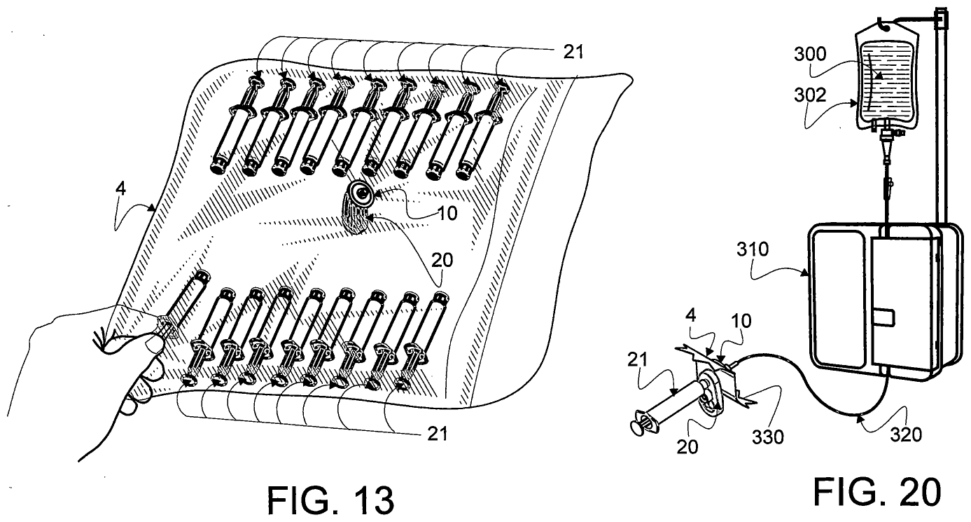

As acquisition and handling free syringes within kit bag 12 can be difficult and, sometimes, frustrating, some helpful methods for handling syringes, such as syringes 21 from outside kit bag 12 is provided. In FIG. 13, note that kit bag 12 can be disposed in a flattened pattern, allowing a syringe 21 to be grasped and readily displaced as seen in FIG. 13.

With like ease, filter assembly 10 can also be grasped from outside kit bag 12 and also can be displaced and rotated. Another mode of syringe acquisition and displacement is seen in FIG. 4 wherein an array 50 of capped, empty syringes (each numbered 21′) is disposed in a tilted tray 52. Geometry of tilted tray 52 disposes syringes 21′ for being readily, individually affixed to capet 20 for displacement and filling as seen by displaced syringe 21″ affixed to capet 20. Thereby devices (i.e. syringe 40′ and a portion of filter assembly 10 which are exterior to plastic bag 12, as seen in FIG. 4, become displacement handles.

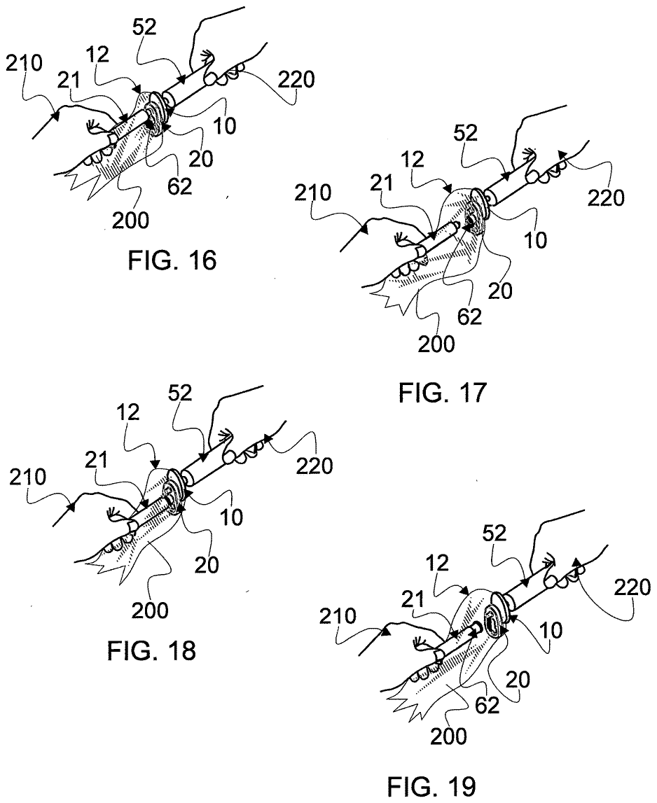

Reference is now made to FIGS. 14-19 wherein decapping and recapping methods are depicted. For clarity of instruction, only a portion 200 of bag 12 is provided, along with characterized hand symbols (hand 210 for a left hand and hand 220 for a right hand) provided as well. As seen in FIG. 14, as a beginning step, cap 56 is inserted into capet 20 orifice 110. In FIG. 15, cap 56 is displaced upward into contact with restraining rim 112 and teeth 114 (see FIG. 9). Thereafter, with cap 56 restrained by capet 20, syringe 21 is displaced for receiving sterilized medicine via filter assembly 10 in FIG. 16.

After filling, syringe 21 is displaced from filter assembly as seen in FIG. 17. Syringe 21 and cap 56 are rejoined as seen FIG. 19. Cap 56 and syringe 21 are displaced downward to free cap 56 (and filled syringe 21) as seen in FIG. 19. Thus each filled and capped syringe can be displaced within kit bag 12 for retrieval, labeling and use.

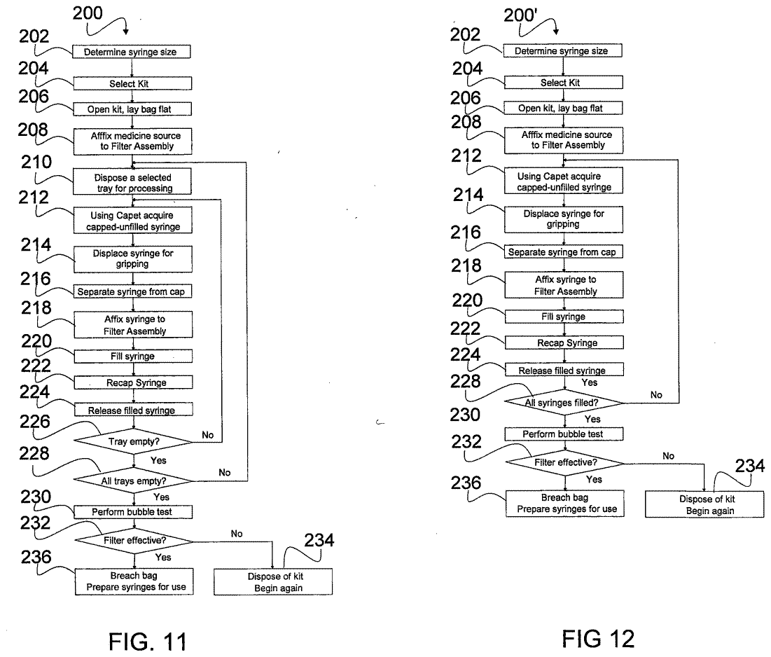

Syringe filling steps, disclosed as examples herein, can involve in two or more modes of use of convenience kits made according to instant inventions disclosed herein. Two such modes are detailed in flow charts provided as FIG. 11 and FIG. 12. The first step identified as function block 202 which determines syringe size for selection of which convenience kit to use. A second step (function block 204) is for acquiring the selected kit from inventory. Kits are generally folded for packaging for shipment, so the next step, see function block 206, is opening the package and preparing kit for use (according to an Instruction for Use, which should be available for all kits). Placing each kit on a flat surface, is important for ease of use.

Note, exemplary variances in kit preparation as depicted in FIGS. 1 and 4 and can range from loose syringes disposed in kit bag 4, as seen in FIG. 1 to syringes in a special tray or trays, as seen in FIG. 4.

Function of block 208 affixes a source of medicine (which can be raw medicine) to a filter assembly 10. An exemplary source of raw medicine and medicine displacement equipment are seen in FIG. 20. In this example the source may include a large bag of raw medicine 300 is provided in a plastic bag container 302. A peristaltic pump 310, preferably provided with electronic controls and an actuating foot actuator peddle (not shown), are affixed via extended medical tubing 320 to a filter assembly 10 which provides sole entry into kit bag 4 seen only in part as section 330. See FIGS. 15-20 for exemplary medicine filling and capping.

Action of function block 210 provides for positioning a tray for empty syringe acquisition. Function of block 212 acquires an empty syringe 21 using a capet 20. See FIG. 4 as an example of such action. Function block 214 prepares for cap 56 removal from syringe 21. See FIGS. 15-18 for cap 56 removal.

Function of block 216 prepares syringe for attachment to filter assembly 10 by removing cap 56. Function of block 218 affixes uncapped syringe 21 to capet 20 and thereby to syringe assembly 10. Function of block 220 fills syringe with a predetermined volume of sterilized medicine. Function of block 222 recaps filled syringe 21, see FIGS. 19 and 20. Function of block 224 dispatches the filled syringe from capet 20 (and filter assembly 10). It is recommended a specific area within kit bag be dedicated to housing filled syringes.

Decision block 226 tests to determine if a tray in use is empty. If so, next step is to function block 210, otherwise next step is decision block 228. Decision block 228 tests whether or not all syringes are filled; if not, next step is selecting and disposing a filled tray for use.

Once all syringes are filled, efficacy of filter 24 must be assured before a step permitting use of filled syringes can be taken. For such, a bubble test is performed as function block 230. Decision block 230 determines the course of use of filled syringes. If the test shows filter 24 efficacy, the next step is breaching bag 4 for access to syringes for labeling and distribution. Filter failure is very uncommon, however, if the test indicates a filter failure, kit bag 4 and all contents are not to be used and are therefore wasted.

It may be noted that action blocks 210, and 226 are not found in FIG. 12 showing steps associated with tray use require more steps than without tray use.

The invention may be embodied in other specific forms without departing from the spirit or essential characteristics thereof. The present embodiment is therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. Method and apparatus for making and providing a convenience kit for filling syringes which are provided presterilized and capped within closed environs of a pre-sterilized plastic bag for being individually decapped and filled with a preselected medicine and recapped for safe transport into a potentially contaminating environment comprising the following steps:
providing said plastic bag;
providing a predetermined number of said presterilized and capped syringes of sufficient capacity for being filled with a desired volume of said medicine;
providing a filter assembly comprising a sterilizing grade filter for being affixed to said plastic bag to provide the only fluid pathway into said plastic bag;
providing a capet for accessing and decapping each of said syringes prior to filling, for holding said cap during filling and for recapping each of said syringes after filling;
providing a filter assembly affixed to said plastic bag to provide the only fluid pathway into said plastic bag and, therefore the only conduit for sterilized medicine into each said syringe;
affixing said filter assembly to said plastic bag;
displacing said capet into said plastic bag;
displacing said capped syringes into said bag;
closing and sealing said plastic bag such that the only pathway into the plastic bag is via said filter assembly; and
sterilizing said plastic bag and contents to a predetermined SAL.

2. A method for using said convenience kit made according to claim 1 comprising the following steps:
providing a source of medicine and affixing said source to said filter assembly exterior to said filter assembly;
a. selecting a capped empty syringe for filling;
b. displacing said selected syringe along with simultaneous engagement of said selected syringe cap by said capet;
c. decapping said selected syringe;
d. displacing said selected syringe for communicating with said filter assembly;
e. filling said selected syringe with a desired volume of medicine;
f. displacing said so-filled selected syringe from said filter assembly
g. further displacing said selected syringe to communicate with said capet;
h. recapping said syringe;
i. freeing said cap from said capet and free said filled and capped syringe from said capet; and
j. if there are more syringes to be filled, proceed to step “a”; otherwise proceed to step k,
k. opening said plastic bag for access to filled and capped syringes for use.

3. The method according to claim 1 wherein said providing step comprises providing said capet securely affixed to said filter assembly.

4. A cap engaging tool (capet), for engaging a syringe cap for thereby decapping and recapping a pre-capped syringe disposed within a closed container in which digital contact with the cap is not available, comprising the following attributes:
an orifice sized and shaped for introduction of said syringe cap;
a cap guide and holding section associated with said orifice whereby said syringe cap is slideably displaced into a cap holding section of said capet;
said cap holding section comprising a cap-rim interface which securely engages said cap whereby said cap is detached from said syringe and retained thereat;
said cap holding section comprising cap access for recapping said syringe; and
said cap guide providing a return pathway to said orifice wherefrom the so-capped syringe is displaced and freed.
